(19) United States
(12) Patent Application Publication
(10) Pub. No.: US 2008/0262408 A1
(43) Pub. Date: Oct. 23, 2008

(54) Multi-Constituent Packaging with Applicator

(76) Inventors: Martin Krauss, Fort Meyers, FL (US); Herbert Schuhrer, Bruchsal (DE)

Correspondence Address: BROWDY AND NEIMARK, P.L.L.C.;624 NINTH STREET, NW, SUITE 300, WASHINGTON, DC, 20001-5303, US

(21) Appl. No.: 11/908,368

(22) PCT Filed: Mar. 7, 2006
(86) PCT No.: PCT/DE2006/000386

§ 371 Date: May 7, 2008

(30) Foreign Application Priority Data
Mar. 11, 2005 (DE) 20 2005 004 135.1

(51) Int. Cl. A61N 1/30; B65D 25/08

(52) U.S. Cl. 604/19; 206/219

(57) ABSTRACT

A packaging for at least two constituents has a tubular or bag-shaped packaging element (1.1, 3.1, 4.1, 5.1, 6.1, 7.1, 8.1, 9.1) and an applicator (1.2, 2.2, 3.2, 4.2, 5.2, 6.2, 7.2, 8.2, 9.2) having a dispensing channel for the mixed constituents and being connectable to the packaging element, wherein the packaging element is designed for storing at least one constituent and for mixing the at least two constituents.

This makes it possible to create a packaging for two or a plurality of constituents that can be mixed together and applied in a simple manner in a metered administration. In a preferred embodiment of the invention the two constituents (or a plurality of constituents) are accommodated jointly in a single flexible (tubular or bag-like) packaging, which also serves as a mixing chamber, whereas the applicator only fulfills its function as an application aid.

### TECHNICAL BACKGROUND

Many substances or mixtures of substances become effective only by being combined or mixed with other substances or mixtures of substances, or they can be properly administered only in their mixed state. In many cases of application this creates the problem that such products cannot be stored in their mixed state or, e.g., they loose their potency. It is therefore advisable to initially store such substances/mixtures of substances separately and to mix them only shortly prior to their application.

Difficulties may arise in this context in that the proper mixing ratio needs to be adhered to, which is of great importance especially in the case of pharmaceutical and chemical products. The open handling of the constituents that are to be mixed can have negative effects particularly if a constituent by itself, for example, has toxic effects, but it is often unadvisable also because certain substances react with the gas mixture of the air and can easily spoil as a result, especially if the constituents are removed from larger packing drums that are stored for an extended period of time after having been opened.

An additional problem point is the proper application method. In applications in the medical field, for example, active substances must be administered in a targeted manner to areas inside the body, e.g., in the nose, vagina or anus, and an applicator that is adapted to the application area facilitates the insertion of the active substance or of the active substance that is embedded in a carrier substance.

However, in other areas as well, such as, e.g. the application of a 2-constituent adhesive, handling is facilitated by an application process that uses an applicator.

### DISCLOSURE OF THE INVENTION

The invention has as its object to create a packaging for two or a plurality of constituents that are to be mixed together and applied in a simple manner in a metered administration, for which an integrated or easily integratable applicator is intended to facilitate the proper handling.

This object is met with the characteristics of claim 1.

A flexible packaging of plastic, aluminum or composite materials thereof contains one or a plurality of constituents that are initially separated from one or a plurality of additional constituents in such a way that a connection between the constituents can be created by simple manipulation, in order to mix the same without significant exposure to the environment. An applicator that is directly integrated into the packaging, or which serves as receptacle for one of the constituents, or which may be designed as a separate part that can be easily affixed to the packaging, facilitates the application of the mixed constituents.

A number of preferred embodiments of the invention are specified in the subclaims, and the following variants in particular will be highlighted below:

The two constituents (or a plurality of constituents) in a preferred embodiment may be accommodated jointly in the flexible (tubular or bag-like) packaging whereas the applicator only fulfills its original function as an administering aid.

According to an additional advantageous embodiment, however the applicator may also perform functions in addition to that, namely it may itself, on one hand, serve as a receptacle volume for a constituent (so that the tubular or bag-like packaging possibly needs to contain only one constituent). In this preferred exemplary embodiment the applicator is preferably designed syringe-like, its inner volume serving on one hand for the initial accommodating of a constituent, but also finding use as a mixing chamber during the operations that are performed up until the application of the mixed product, in such a way that a repeated exchange of the product or mixture between the interior of the applicator of this design on one hand, and the interior of the flexible packaging takes place by means of the syringe plunger.

The exemplary embodiments that will be described below, will in turn illustrate a multitude of design combinations for both variants, with respect to the design of the tubular or bag-like packaging element on one hand and of the applicator on the other hand.

The applicator may, for example, also be constructed of multiple components to provide the above-described functions for joining it to the flexible packaging, optionally for accommodating a constituent, and for mixing the constituents, as well as for dispensing the mixed product in the respective product-specific optimal form.

### BRIEF DESCRIPTION OF THE FIGURES

A number of exemplary embodiments of the inventive multi-constituent packaging will be explained in more detail based on drawings:

FIG. 1 shows a first exemplary embodiment of the multi-constituent packaging with applicator,

FIG. 2 shows the activation and use of the multi-constituent packaging according to the first exemplary embodiment,

FIG. 3 shows sections through the tubular packaging element with possible embodiments,

FIGS. 4 through 11 show various embodiments of the joining region between the applicator and tubular packaging element,

FIG. 12 through FIG. 19 show variations of the applicator in sectional views,

FIG. 20 shows a second exemplary embodiment of the multi-constituent packaging with applicator in a full sectional view,

FIG. 21 shows a third exemplary embodiment of the multi-constituent packaging with applicator in a full sectional view,

FIG. 22 shows a fourth exemplary embodiment of the multi-constituent packaging with applicator in a full sectional view,

FIG. 23 shows a fifth exemplary embodiment of the multi-constituent packaging with applicator in a full sectional view,

FIG. 24 shows a sixth exemplary embodiment of the multi-constituent packaging in a full sectional view,

FIG. 25 shows a seventh exemplary embodiment of the multi-constituent packaging in a full sectional view, and

FIG. 26 and FIG. 27 show two embodiments of the joining region of the applicator to the tubular packaging element in a partial section and in a front elevation.

### DESCRIPTION OF THE EXEMPLARY EMBODIMENTS

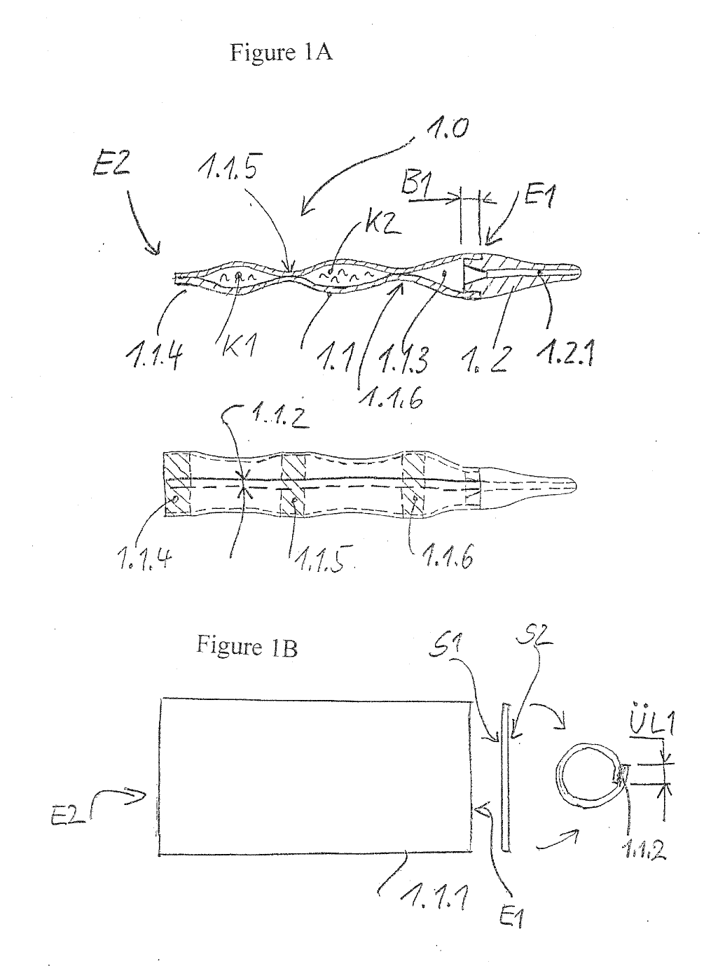

FIG. 1A shows a first exemplary embodiment, a multi-constituent packaging 1.0 in a full sectional view and in a top view. FIG. 1B shows a starting format for the tubular packaging element.

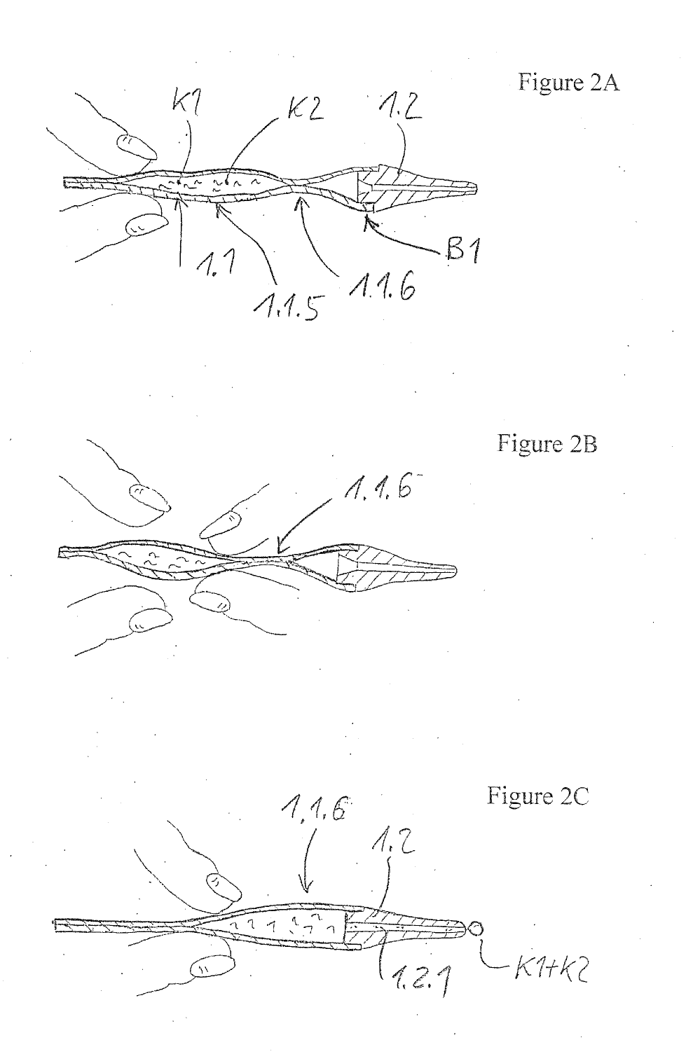

FIGS. 2A,2B,2C show the activation and use of the multi-constituent packaging 1.0.

The multi-constituent packaging 1.0 consists of a tubular packaging element 1.1 as the basic element and of an applicator 1.2. The foundation for the tubular packaging element 1.1 is a flexible foil 1.1.1 (a tubular extruded piece may be used as well) of plastic, Al, or their composite materials, which may be laminated or coated, depending on the intended use.

In the first exemplary embodiment the packaging element is created through an overlap ÜL1 of the longitudinal sides of the foil 1.1.1. The foil 1.1.1 may be sealably coated on its side S1 and also on its side S2, in such a way that when certain parameters are adhered to such as heat or pressure during the overlapping or layering of the foil 1.1.1, a firm or also a peelable adhesion at the contact surfaces can be achieved.

This packaging element 1.1 in the first exemplary embodiment is thus produced from a single flat foil 1.1.1, which takes its tubular shape through a firm joining region 1.1.2 (overlap ÜL) extending in the main out direction.

The tubular packaging element 1.1, which is initially open at the end regions E1 and E2, is firmly joined at its end E1 to an applicator 1.2 which preferably consists of plastic, in the region B1, e.g., by sealing or welding, with the material and shape of the applicator 1.2 matching the joining method and area of application.

The applicator 1.2 has a preferably centrical continuous dispensing channel 1.2.1 forming a connection to the interior 1.1.3 of the frontal end region E1 of the tubular packaging element 1.1. The rear end region E2 of the tubular packaging element is firmly surface-bonded in the region 1.1.4 e.g., by sealing. Extending between the firm joining region 1.1.4 at the end region E2 and the region of the applicator 1.2 is at least one separable joining area 1.1.5 for separation of the constituents K1 and K2 that are to be mixed, and optionally one additional separable joining area 1.1.6 near the applicator. The joining area 1.1.6 bounds the region of the constituent K2 so that the same cannot inadvertently migrate into the applicator 1.2 or to the outside. The constituents K1 and K2 may be either liquid, pasty, powdery, or granular; and combinations thereof, such as, e.g., a liquid constituent combined with a powdery constituent, are possible as well.

To mix the constituents K1 and K2, pressure is exerted via the tubular packaging element 1.1 onto one of the constituents in the direction of the other constituent, causing the joining area 1.1.5 between the two constituents K1 and K2 to open. By alternating pressure onto the tubular packaging element 1.1 in the region of the constituents K1 and K2 one causes them to be mixed together.

Further pressure, or squeezing the mixed constituents out in the direction of the applicator 1.2, causes the joining area 1.1.6 to separate. The packaged contents travel via the dispensing channel 1.2.1 of the applicator 1.2 to the outside and are placed at the intended location by means of the applicator 1.2.

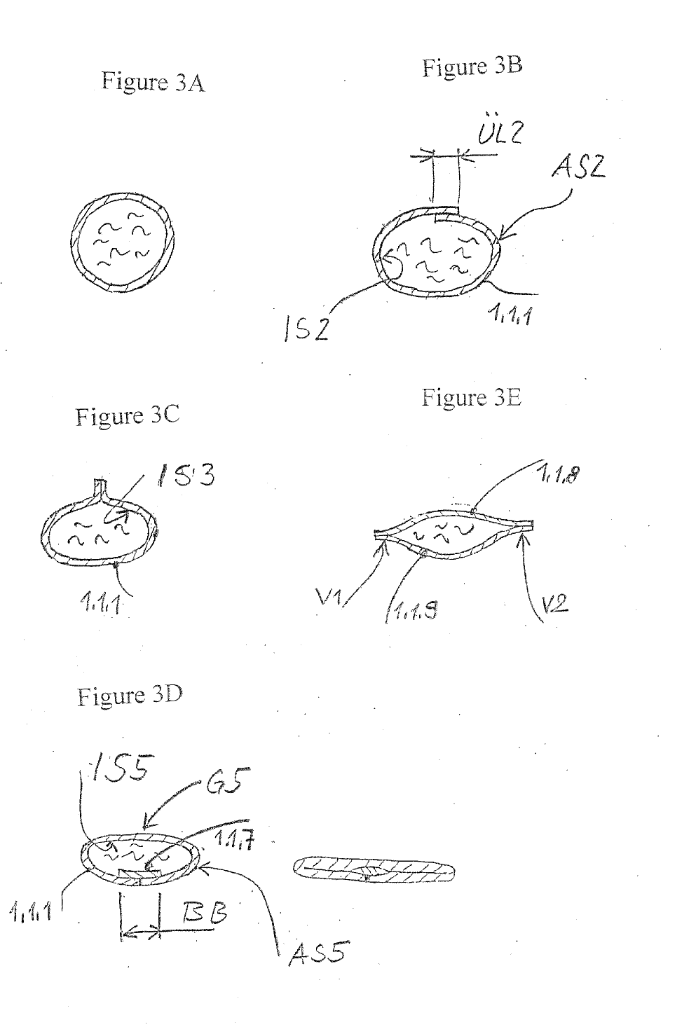

FIG. 3 shows sections through the tubular packaging element depicting possible embodiments:

In FIG. 3A a tubular packaging element is shown without overlap, as it is the case with a production from an extruded tube.

FIG. 3B shows a section through a tubular packaging element with a single overlap ÜL2, as it has already been described in the first exemplary embodiment, wherein the inside surface IS2 comes into contact with the outside surface AS2 of the foil 1.1.1.

FIG. 3C shows a section through a tubular packaging element, wherein the longitudinal edges of what will later become the inside surface IS3 of the foil 1.1.1, are placed together for joining and firmly joined in the main out direction of the multi-constituent packaging.

FIG. 3D shows a section in each case through an additional tubular packaging element in the region of the packaged contents and in the region of the surface-bonded seal connection. The foil 1.1.1 is butt-jointed in this case, the tight bond is implemented by means of an additional sealable strip 1.1.7 having the strip width BB at what will later become the inside surface IS5 of the basic element. The strip 1.1.7 is preferably joined to the foil 1.1.1 in the main out direction of the packaging by welding or sealing. The strip 1.1.4 may optionally also be affixed on the outside surface AS5 of the foil 1.1.1.

FIG. 3E shows a section through a tubular packaging element, the foil in this case consists of two individual foils 1.1.8 and 1.1.9, so that the tubular packaging element is provided with two joining regions V1 and V2 extending in the main out direction of the packaging.

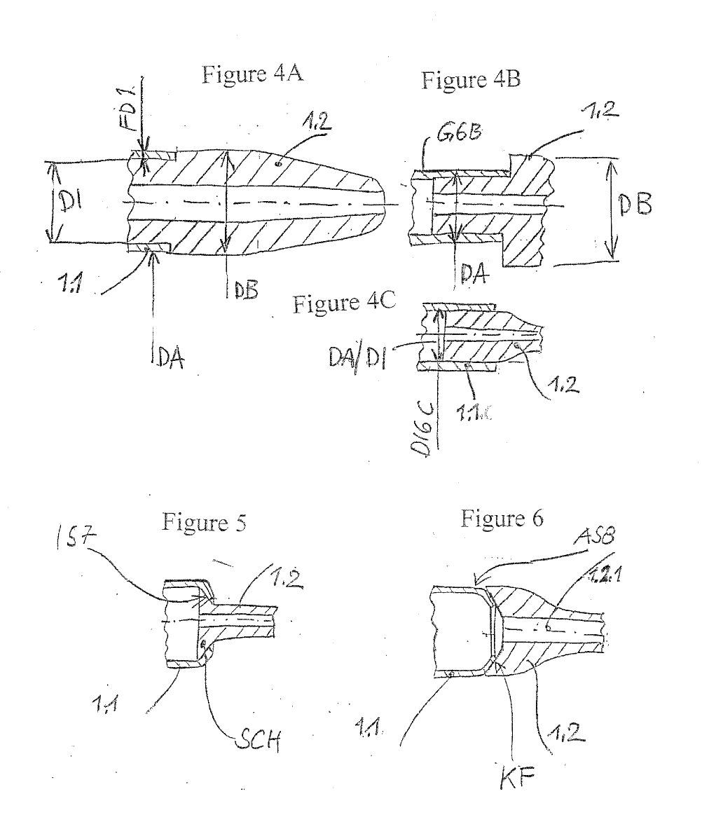

FIGS. 4 through 11 show different embodiments of the joining region between the applicator 1.2 and the tubular packaging element 1.0:

In FIG. 4A the outside diameter DA of the tubular packaging element 1.1 is approximately identical to the outside diameter DB of the applicator in the joining region, the applicator 1.2 being provided with an annular shoulder whose diameter DI is smaller by approximately double the foil thickness FD1 than the outside diameter DA of the tubular packaging element 1.1.

In FIG. 4B the outside diameter DA of the tubular packaging element 1.1 is significantly smaller than the outside diameter DB of the applicator 1.2 in the joining region,

in FIG. 4C the inside diameter DI of the packaging element 1.1 is identical to the outside diameter DA of the applicator 1.2 in the joining region,
in FIG. 5 the applicator 1.2 is implemented such that it expands in the direction of the tubular packaging element 1.1 in the form of a shoulder SCH, so that the inside surface IS7 of the tubular packaging element 1.1 can be flanged over the shoulder region SCH of the applicator 1.2 and joined to the same preferably by sealing or welding,
in FIG. 6 the applicator 1.2 is implemented such that the dispensing channel widens in the direction of the tubular packaging element 1.1 in a cone shape KF, so that the outside surface AS8 of the basic tubular element can be flanged into it and joined to the applicator 1.2 preferably by sealing or welding.

In FIG. 7 the tubular packaging element 1.1 is depicted as a composite of two foils 1.1.8 and 1.1.9, but it can also be implemented with basic tubular elements that are joined in the main out direction in the variants described so far. The cross section SF9 of the joining section VB9 of the applicator 1.2 is designed here in the shape of a boat that is pointed at both ends, the foils 1.1.8 and 1.1.9 projecting beyond the joining section SF9 on both sides by a dimension M9, so that their projecting inside surfaces IS9A and (S9B can be joined, e.g., by sealing.

In FIG. 8 the applicator has been replaced by a an applicator joining piece 1.3 having a boat-shaped cross section and conical opening 1.3.1, which tapers toward the tubular packaging element 1.1. This opening serves for docking of a withdrawal device (which is not depicted here). The applicator joining piece 1.3 is sealed with an original-quality seal, e.g., in the form of a peelable foil 1.3.4.

In FIG. 9 the applicator joining piece 1.3 is designed rotation-symmetrical.

In FIG. 10 the opening 1.3.1 of the applicator joining piece has an inside thread 1.3.2.

In FIG. 11 the applicator joining piece 1.3 has a region 1.3.6 that is lengthened with an outside thread 1.3.5, allowing the seal to be implemented also by means of a suitably designed matching cap 1.3.7.

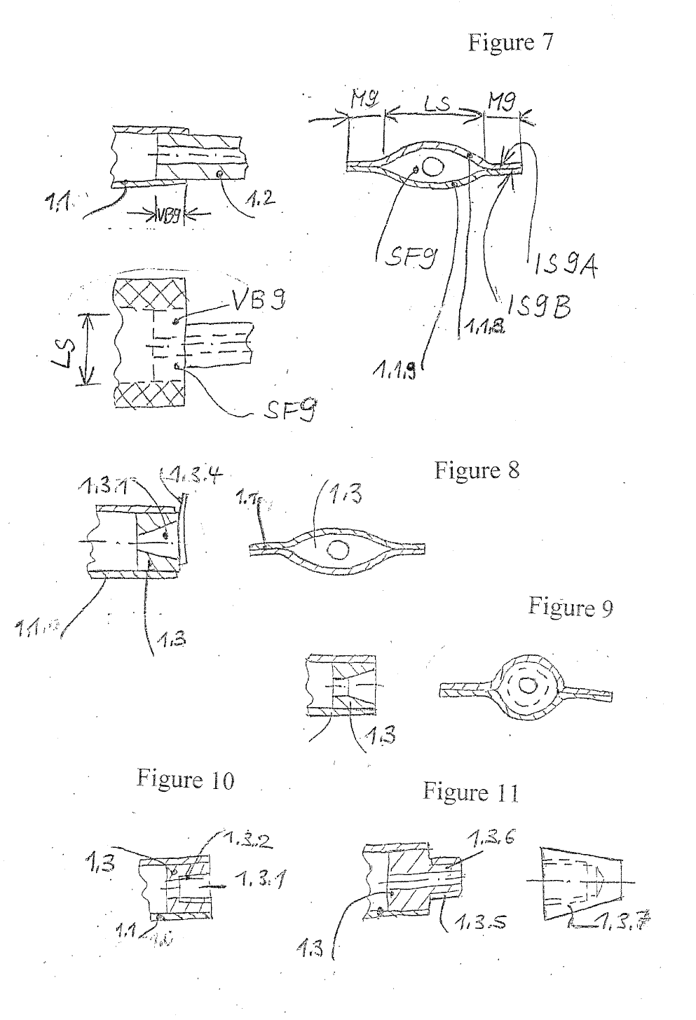

FIG. 12 through FIG. 19 show variations of the end region of the applicator 1.2 in sectional views. All applicators have in their center a continuous dispensing channel for the packaged contents to pass through.

In FIG. 12 the applicator tapers toward the product discharge opening PA1 and is widened at the product discharge opening PA1 by a rounded knot K1, thereby reducing the risk of injury during insertion into orifices of the body.

In FIG. 13 the applicator tapers toward the product discharge opening PA2 and ends in a rounded region R2.

In FIG. 14 the applicator tapers toward its end and ends in a rounded region R3. The product discharge opening is provided in the wall WA3 of the applicator in the form of multiple product discharge openings PAX.

In FIG. 15 the applicator tapers toward its end and ends in a flat shape in the style of a spatula SP4.

In FIG. 16 the applicator is designed as a separate part, with an inside thread 1.2.2 for docking to a packaging 1.8 that has a threaded joining piece.

In FIG. 17 the applicator is also designed as a separate part, with an outside thread 1.2.3 for docking to a packaging that has a threaded joining piece.

In FIGS. 18 and 19 a predetermined break line SB7 is formed in the applicator in the direction of the product discharge opening by means of a weakening in the material. The end of the applicator is widened to form a wing-like end piece 1.2.4. The channel 1.2.1 in the interior of the applicator terminates in the wing-like end piece 1.2.4, which is broken off prior to the application, so that the channel 1.2.1 is opened and the contents of the packaging can be applied to the application area.

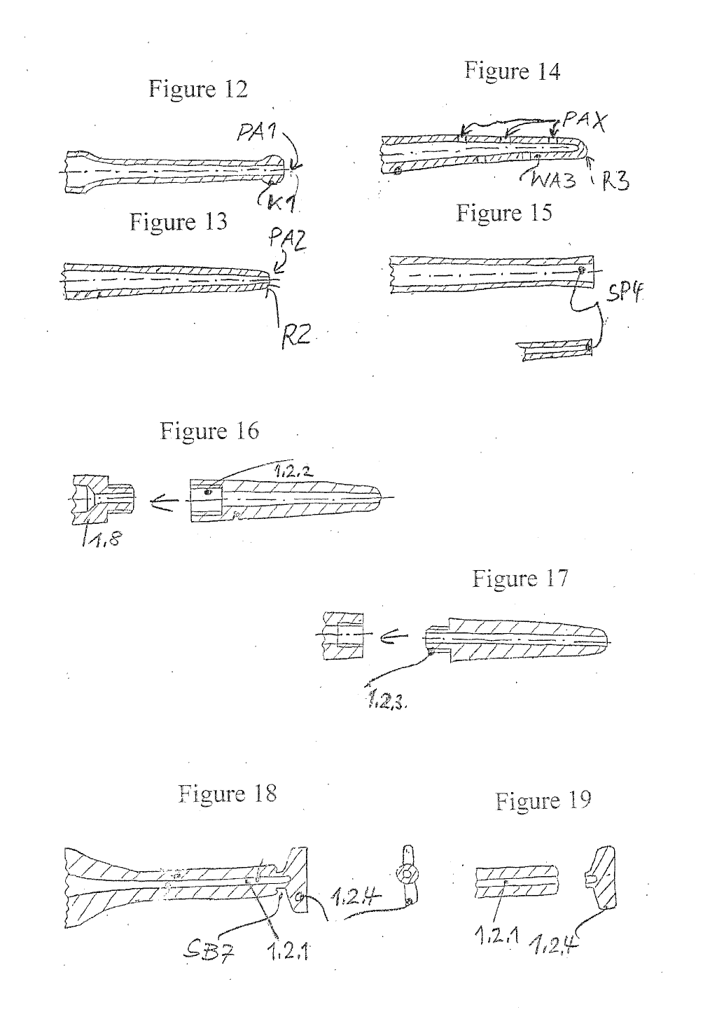

FIG. 20 shows a second exemplary embodiment, a multi-constituent packaging 2.0 with applicator in a full sectional view. In FIG. 20A the packaging is shown not activated. FIG. 20B shows the applicator after drawing in of the mixed constituents. FIG. 20C shows the application of the mixed constituents.

The multi-constituent packaging 2.0 consists of an applicator joining piece 2.1 with cone-shaped docking area 2.1.1 as described under FIG. 8, and of an applicator 2.2. The basic design of the applicator 2.2 corresponds to the basic design of a syringe and will not be described in detail. The end region 2.2.1 of the applicator is rounded and adapted to the cone-shaped docking area 2.1.1 of the applicator joining piece 2.1.

After mixing of the constituents K3 and K4 of the multi-constituent packaging in the previously described manner (not depicted) in the packaging element 1.0, the original-quality seal 2.1.2 is peeled off from the applicator joining piece 2.1 and the applicator 2.2 is docked on. By pulling back the plunger 2.2.2, the mixed constituents K3 and K4 are drawn into the applicator 2.2 and placed at the application area by pushing in the plunger 2.2.2.

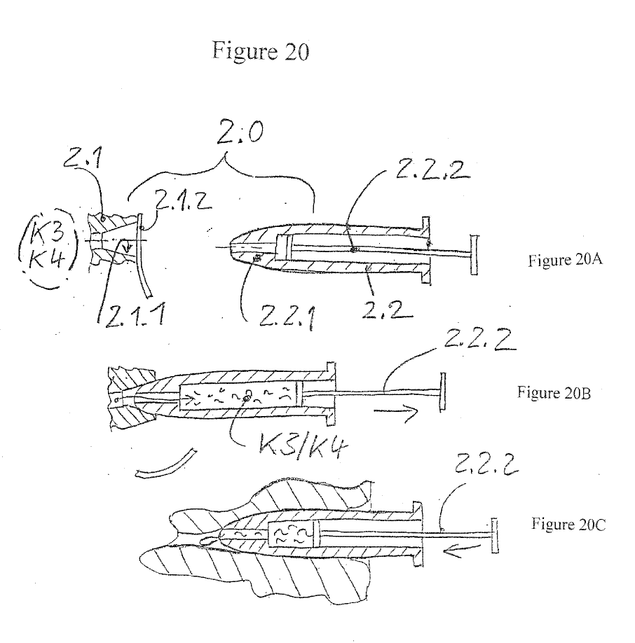

FIG. 21 shows a third exemplary embodiment, a multi-constituent packaging 3.0 with applicator in a full sectional view. In FIG. 21A the packaging is shown not activated. FIG. 21B shows the applicator drawing in the mixed constituents K5 and K6. FIG. 21C shows an applicator attachment being attached. FIG. 21D shows the application of the mixed constituents.

The multi-constituent packaging 3.0 consists of a packaging element 3.1 having an inside thread 3.1.1 as a docking means, as described above, and of the applicator 3.2. The basic design of the applicator corresponds to the basic design of a syringe and will not be described in detail. The end region 3.2.1 of the applicator is provided with an outside thread.

After mixing of the constituents K5 and K6 of the multi-constituent packaging in the previously described manner the applicator 3.2 is docked on. By pulling back the plunger 3.2.2 the mixed constituents K5 and K6 are drawn into the applicator. Depending on the area of application, the application may now take place directly or by means of an applicator attachment 3.3 suitable for the application.

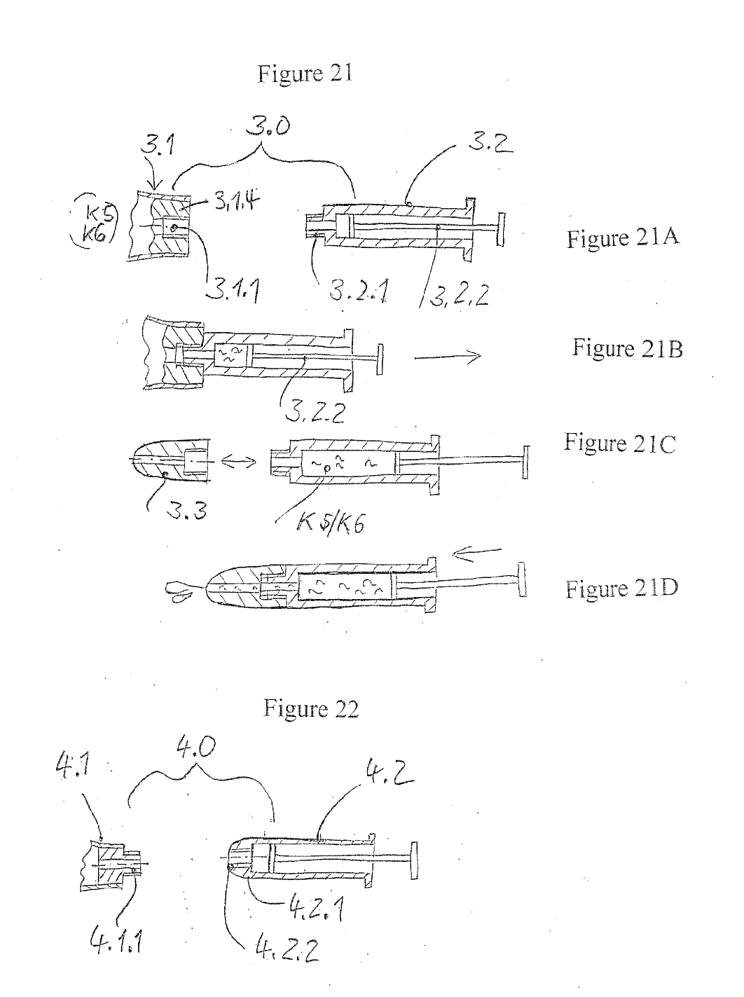

FIG. 22 shows a fourth embodiment, a multi-constituent packaging 4.0 in a full sectional view. The multi-constituent packaging 4.0 consists of a packaging element 4.1 having an outside thread 4.1.1 as a docking means, as it has been described above, and of the applicator 4.2. The basic design of the applicator 4.2 corresponds to the basic design of a syringe and will not be described in detail. The end region 4.2.1 of the applicator is provided with an application-specific shape and has an inside thread 4.2.2. The application corresponds to that in the exemplary embodiment 2.0.

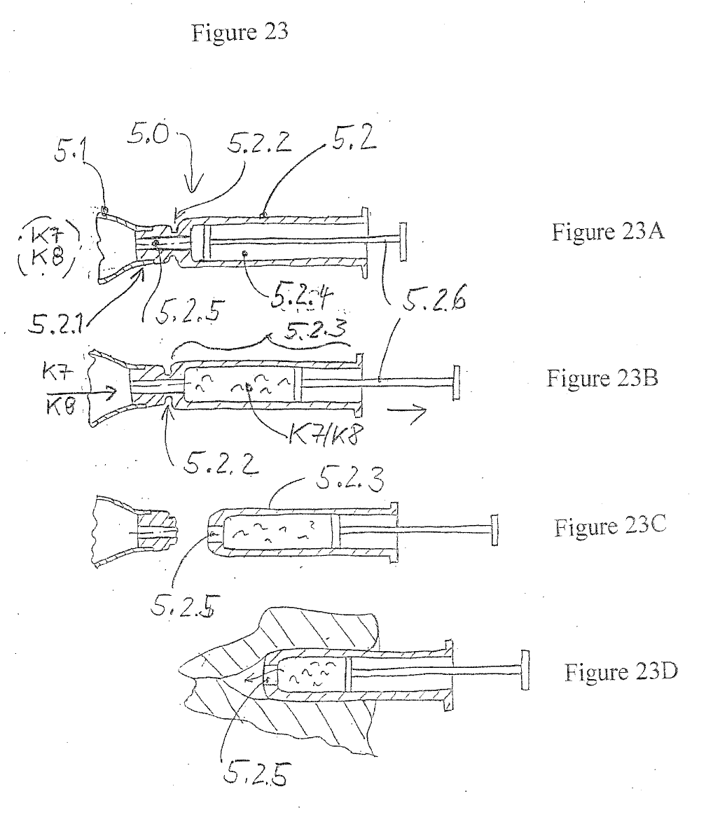

FIG. 23 shows a fifth embodiment, a multi-constituent packaging 5.0 in a full sectional view. FIG. 23A shows the packaging not activated. FIG. 23B shows the mixed constituents being drawn in. FIG. 23C shows the opened packaging. FIG. 23D depicts the application of the contents.

The multi-constituent packaging 5.0 consists of a preferably tubular packaging element 5.1 for accommodation of the constituents K7 and K8 to be mixed, as described under the first exemplary embodiment, and of the applicator 5.2. The joining region 5.2.1 between the tubular packaging element 5.1 and applicator 5.2 may be designed in the previously described variants. The applicator 5.2 is designed in the form of a syringe and embodied in its end region in the form of the joining region 5.2.1. The joining region 5.2.1 has a weakening 5.2.2 in the material as the predetermined break line. The hollow syringe chamber 5.2.4 is connected to the tubular packaging element 5.1 via a tubular hollow space 5.2.5 that extends through the joining region 5.2.1.

After mixing of the constituents K7 and K8 in the previously described manner the packaged contents are drawn into the applicator 5.2 by pulling back the syringe plunger 5.2.6 and the applicator is opened in the joining region 5.2.1 by breaking it open at the weakening 5.2.2 in the material so that the mixed packaged contents K7 and K8 can be applied through the remaining tubular hollow space 5.2.5.

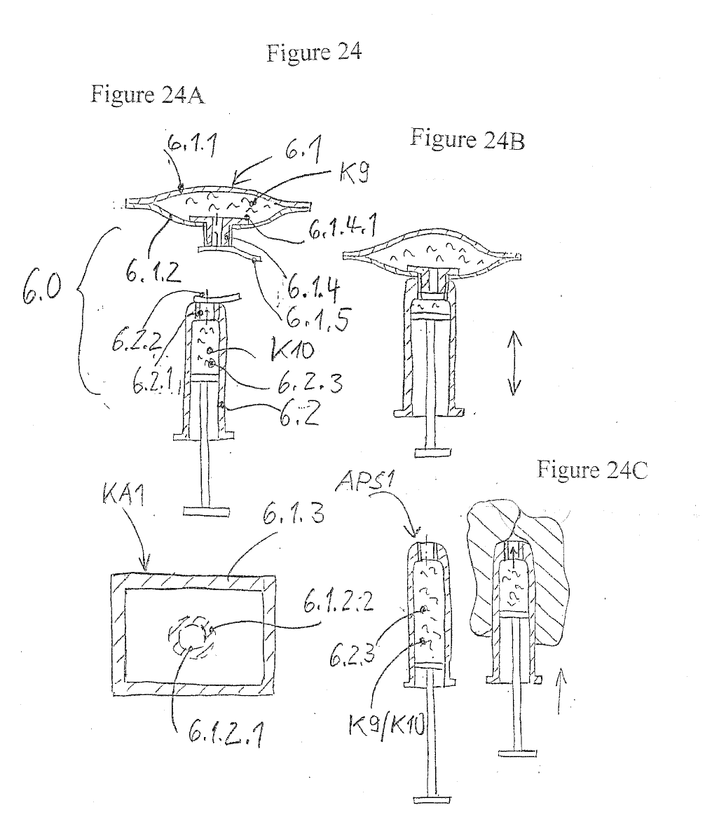

FIG. 24 shows a sixth exemplary embodiment, a multi-constituent packaging 6.0. FIG. 24A shows the sealed packaging in a full sectional view and in a top view. FIG. 24B shows a full sectional view through the packaging during mixing of the constituents. FIG. 24C shows the contents of the packaging being applied.

The multi-constituent packaging 6.0 consists of a bag-like packaging element 6.1 for accommodation of the constituent K9 and of the applicator 6.2 in the form of a syringe, which, in this case as well, serves for accommodation of an additional constituent K10 as well as for mixing of the constituents. The packaging element 6.1 consists of foils 6.1.1 and 6.1.2 that are preferably sealable on one side, which are joined to each other along the periphery in the region 6.1.3 preferably by sealing. The outer contour KA1 may be designed in any suitable shape. The foil 6.1.2 is provided with a punched-out area to receive a nipple 6.1.4 provided with an outside thread of sealable material. The nipple 6.1.4 is enlarged in its end region in the form of a flange 6.1.4.1 and inserted into the punched-out area 6.1.2.1 in such a way that it protrudes through the packaging element 6.1 to the outside, the flange 6.1.4.1. resting against the sealable surface of the foil 6.1.2 and being joinable to the same in the region 6.1.2.2 preferably by sealing. The packaging element 6.1 can be sealed, e.g., by sealing-on a peelable foil 6.1.5 in the region of the threaded nipple 6.1.4, or with the aid of a cap.

The applicator 6.2 for accommodation of the constituent K10 is provided at what will later become its application side APSI with an inside thread 6.2.1 and is sealed, e.g., by means of a sealed-on peelable foil 6.2.2 or by means of a screw-type element. For mixing the two constituents K9 and K11, the peelable foils 6.1.5 and 6.2.2 are peeled off from the end region of the applicator 6.2 or nipple 6.1.4 of the packaging element 6.1. Afterwards the packaging element 6.1 and applicator 6.2 are brought together in the region of their threads. By pressing the constituents K10 into the packaging element 6.1 or drawing the constituent K9 out into the hollow chamber 6.2.3 of the applicator 6.2 with repeated exchange of the partially mixed constituents from the packaging element 6.1 into the applicator 6.2 and from the applicator 6.2 into the packaging element 6.1, respectively, mixing of the constituents K9 and K10 takes place. After the mixing, the constituents K9 and K10 are drawn into the applicator 6.2, and the packaging element 6.1 and applicator 6.2 are separated so that the contents can be applied.

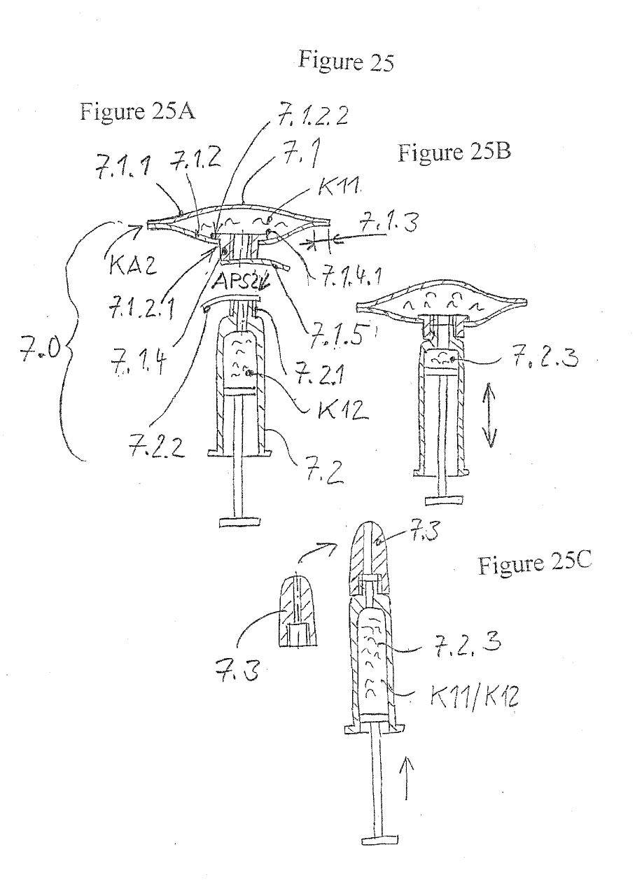

FIG. 25 shows a seventh exemplary embodiment, a multi-constituent packaging 7.0. FIG. 25A shows the sealed packaging in a full sectional view and in a top view. FIG. 25B shows a full sectional view through the packaging during mixing of the constituents. FIG. 25C depicts the packaging prior to the application of its contents.

The multi-constituent packaging 7.0 consists of a packaging element 7.1 in the form of a bag for accommodation of the constituent K11 and of an applicator 7.2 in the form of a syringe for accommodating a further constituent K12. The packaging element 7.1 consists of foils 7.1.1 and 7.1.2 that are preferably sealable on one side, which are joined to each other along their periphery in the region 7.1.3 preferably by sealing. The outer contour may be designed in any suitable shape. The foil 7.1.2 is provided with a punched-out area 7.1.2.1 for receiving a nipple with an inside thread 7.1.4 of sealable material. The nipple 7.1.4 is enlarged in its end region in the form of a flange 7.1.4.1 and inserted into the punched-out area 7.1.2.1 in such a way that is protrudes through the packaging element 7.1 to the outside and that its flange 7.1.4.1 rests against the sealable surface of the foil 7.1.2 and can thus be joined to the same in the region 7.1.2.2 preferably by sealing.

The packaging element 7.1 may be sealed e.g., by sealing-on a peelable foil 7.1.5 in the region of the nipple 7.1.4 or with the aid of a screw-type element. The applicator 7.2 for accommodation of the constituent K12 is provided at what will later become its application side APS2 with an outside thread 7.2.1 and is sealed, e.g., by means of a sealed-on peelable foil 7.2.2 or by means of a cap. For mixing the two constituents K11 and K12, the peelable foils 7.2.2 and 7.1.5 are peeled off from the applicator 7.2 or nipple 7.1.4 of the packaging element 7.1. Afterwards the packaging element 7.1 and 7.2 are brought together in the region of their threads. By pressing the constituents K12 into the packaging element 7.1 or drawing the constituent K11 out into the applicator 7.2 with repeated exchange of the partially mixed constituents from the packaging element 7.1 into the hollow applicator space 7.2.3 and from the hollow applicator space 7.2.3 into the packaging element 7.1, respectively, mixing of the constituents K11 and K12 takes place. After the mixing, the constituents K11 and K12 are drawn into the applicator 7.2, and the packaging element 7.1 and applicator 7.2 are separated so that the contents can be applied, optionally by attaching an application-specific shaped piece 7.3.

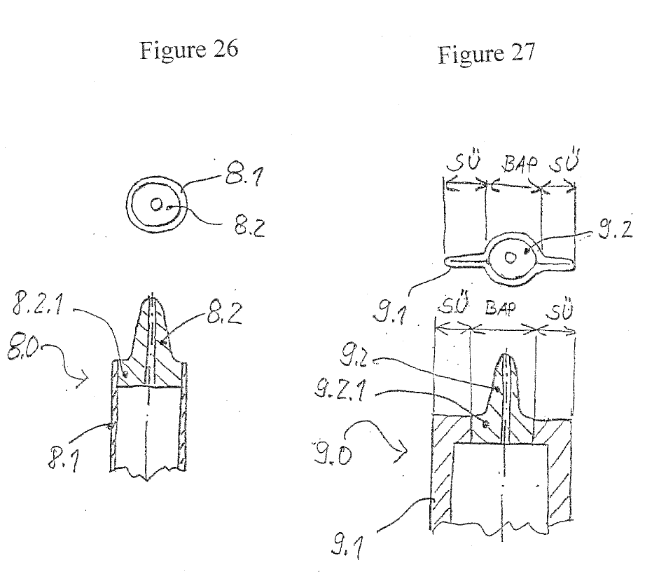

FIG. 26 and FIG. 27 show embodiments of the joining region of the applicator to the tubular packaging element in a partial section, as well as the bottom plan view.

For all packaging variations in which the applicator is connected to the tubular region in the longitudinal out direction, the tubular packaging element 8.1 may be selected in its dimensions such that it may be joined with the joining region 8.2.1 of the applicator 8.2 without laterally projecting excess material (FIG. 26), or like in the exemplary embodiment 9.0, such that it is larger than the joining region 9.2.1 of the applicator 9.2, so that it projects in the region SÜ beyond the joining region 9.2.1 by the dimensions BAP on one or both sides. The position of a joining region of the tubular packaging element, which optionally extends in the main out direction (shown in the first exemplary embodiment), may take different positions depending on the joining method and later function.

1-13. (canceled)

14. A packaging for at least two constituents, having a packaging element (1.1), one end (E2) of which is sealed with a surface-bonded firm joining region (1.1.4) and the other end (E1) of which is open for accommodating an applicator (1.2), the packaging element having disposed over its length at least two joining regions (1.1.5), (1.1.6) for creation of at least two compartments (K1, K2) for storing and for mixing of the at least two constituents, which can be selectively opened by exertion of pressure to effect the mixing of the at least two constituents and dispensing thereof to the applicator (1.2), which has a dispensing channel for the mixed constituents, and one end of which is firmly joined to the open end of the packaging element (1.1) especially by sealing or welding, characterized in that the packaging element is formed of a tube and that the applicator (1.2) consists of at least one rotation-symmetrical shaped member, the other end of which is designed preferably conical for insertion into an organ.

15. A packaging according to claim 14, characterized in that the tube consists of a cut (1.1.1) of plastic foil, metal foil, or foil of composite materials that is joined along its longitudinal sides.

16. A packaging according to claim 14, characterized in that the applicator (1.2) consists of a coupling element (1.8) for joining it to the tube, the coupling element containing the first end of the applicator, and a of dispensing element (1.2.2), which incorporates the second end for insertion into an organ, and that the coupling element (1.8) and dispensing element (1.2.2) are connectable to each other in a form-fitting or frictional manner.

17. A packaging according to claim 14, characterized in that the dispensing channel (1.2.1) is sealed in the second end of the applicator, and a predetermined break line (SB7) is provided in the region of the dispensing channel for exposing a dispensing opening.

18. A packaging according to claim 15, characterized in that the dispensing channel (1.2.1) is sealed in the second end of the applicator, and a predetermined break line (SB7) is provided in the region of the dispensing channel for exposing a dispensing opening.

19. A packaging according to claim 16, characterized in that the dispensing channel (1.2.1) is sealed in the second end of the applicator, and a predetermined break line (SB7) is provided in the region of the dispensing channel for exposing a dispensing opening.
